(19) United States
(12) Patent Application Publication
(10) Pub. No.: US 2011/0152187 A1
(43) Pub. Date: Jun. 23, 2011

(54) INSULIN-LIKE GROWTH FACTOR FUSION PROTEINS

(75) Inventors: Peter Artymiuk, Sheffield (GB); Richard Ross, Sheffield (GB); Jon Sayers, Sheffield (GB)

(73) Assignee: ASTERION LIMITED, Sheffield (GB)

(21) Appl. No.: 12/672,000

(22) PCT Filed: Aug. 5, 2008
(86) PCT No.: PCT/GB08/02655

§ 371 Date: Mar. 10, 2011

Related U.S. Application Data
(60) Provisional application No. 60/956,333, filed on Aug. 16, 2007.

(30) Foreign Application Priority Data
Aug. 6, 2007 (GB) 0715213.5

(51) Int. Cl. A61K 38/30; C07K 14/475; C12N 15/18; C12N 15/63; C12N 5/00; A61P 5/00; A61P 3/10; A61P 17/02

(52) U.S. Cl. 514/8.5; 530/399; 536/23.5; 435/320.1; 435/325

(57) ABSTRACT

This disclosure relates to insulin-like growth factor fusion polypeptides; nucleic acid molecules encoding said polypeptides and methods of treatment that use said polypeptides.

The invention relates to insulin-like growth factor fusion polypeptides and dimers; nucleic acid molecules encoding said polypeptides and methods of treatment that use said polypeptides/dimers.

IGF-1 is a 70 amino acid polypeptide with a molecular weight of 7.6 kD. IGF-1 stimulates, amongst other cells, the proliferation of chondrocytes resulting in bone growth. IGF-1 is also implicated in muscle development. IGF-1 is an example of a protein ligand that interacts with members of the receptor tyrosine kinase (RTK) superfamily. Approximately 98% of IGF-1 is bound to one of six binding proteins (IGF-BP). IGF-BP3 is the most abundant and accounts for 80% of IGF-1 binding. IGF-1 binds two receptors; the IGF-1 receptor (IGFR) and insulin receptor (IR) the former of which is bound with greater affinity. In addition to IGF1R, other members of the RTK superfamily include the insulin receptor (IR), epidermal growth factor receptor (EGFR, also known as ErbB1) and related ErbB receptors, vascular endothelial growth factor receptor (VEGF) and nerve growth factor (NGFR). The extracellular domains each consist of several subdomains of a variety of different architectures including immunoglobulin-like, cysyteine-rich, EGF-like. As with the cytokine receptor family, activation is thought to occur through ligand-mediated oligomerization, in most cases probably by dimerization.

Other hormone systems can interact with IGF-1 signalling. For example, human growth hormone, also known as somatotropin, is a protein hormone/cytokine of about 190 amino acids and is synthesized and secreted by the cells of the anterior pituitary. It functions to control several complex biological processes including growth and metabolism. Growth hormone can have direct effects through binding growth hormone receptor expressed by responsive cells and indirect effects which are primarily mediated by insulin-like growth factor (IGF-1). A major role of growth hormone is therefore the stimulation of the liver to produce IGF-1.

Pathologies that result from a lack of IGF-1 production or IGF-1 insensitivity are known in the art. An example of a severe primary IGF-1 deficiency is Laron dwarfism. The disease does not respond to growth hormone therapy since suffers do not express growth hormone receptor. Other forms of severe primary IGF-1 deficiency include sufferers that carry growth hormone receptor mutations and mutations in IGF-1 or IGFR. In addition recombinant IGF-1 has been evaluated in the treatment of a number of conditions, for example type I and type II diabetes, amyotrophic lateral sclerosis, severe burn injury and myotonic muscular dystrophy. It is also known that IGF-1 has a role in the maintenance of tumours and therefore IGF-1 antagonists will have therapeutic value in the treatment of cancer.

This disclosure relates to the identification of IGF-1 recombinant forms that have improved pharmacokinetics (PK) and activity. The new IGF-1 molecules have biological activity, form dimers and have improve stability.

According to an aspect of the invention there is provided a nucleic acid molecule comprising a nucleic acid sequence that encodes a polypeptide that has the activity of insulin-like growth factor comprising an insulin-like growth factor polypeptide linked, directly or indirectly, to at least one binding domain of insulin-like growth factor receptor.

According to an aspect of the invention there is provided a fusion polypeptide comprising: the amino acid sequence of insulin-like growth factor polypeptide, or active part thereof linked, directly or indirectly, to at least one insulin-like growth factor polypeptide binding domain of the insulin-like growth factor polypeptide receptor polypeptide.

In a preferred embodiment of the invention said polypeptide binding domain comprises or consists of a leucine rich amino acid motif.

In a preferred embodiment of the invention said leucine rich amino acid motif comprises or consists of amino acids 31-179 of SEQ ID NO: 14.

In an alternative preferred embodiment of the invention said leucine rich amino acid motif comprises or consists of amino acids 229-487 of SEQ ID NO: 14.

In a further preferred embodiment of the invention said polypeptide comprises at least one fibronectin III binding domain; preferably said domain comprises or consists of the amino acid residues 494-606 of SEQ ID NO: 14.

In a preferred embodiment of the invention insulin-like growth factor polypeptide is linked to the leucine rich binding domain wherein said insulin-like growth factor polypeptide is positioned amino-terminal to said leucine rich domain in said fusion polypeptide.

In alternative preferred embodiment of the invention insulin-like growth factor polypeptide is linked to the leucine rich binding domain wherein said insulin-like growth factor polypeptide is positioned carboxyl-terminal to said leucine rich domain in said fusion polypeptide.

In a preferred embodiment of the invention insulin-like growth factor polypeptide is linked to the fibronectin III binding domain wherein said insulin-like growth factor polypeptide is positioned amino-terminal to said fibronectin III binding domain in said fusion polypeptide.

In an alternative preferred embodiment of the invention insulin-like growth factor polypeptide is linked to the fibronectin III binding domain wherein said insulin-like growth factor polypeptide is positioned carboxyl-terminal to said fibronectin III binding domain in said fusion polypeptide.

In a preferred embodiment of the invention insulin-like growth factor polypeptide is linked to the leucine rich domain of the insulin-like growth factor receptor polypeptide by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5, 6, 7, 8, 9 or 10 copies of the peptide Gly Gly Gly Gly Ser.

In an alternative embodiment of the invention said polypeptide does not comprise a peptide linking molecule and is a direct fusion of insulin-like growth factor polypeptide and the leucine rich binding domain of the insulin-like growth factor receptor polypeptide.

According to an aspect of the invention there is provided a nucleic acid molecule comprising a nucleic acid sequence selected from:
i) a nucleic acid sequence as represented in SEQ ID NO: 1;
ii) a nucleic acid sequence as represented in SEQ ID NO:3;
iii) a nucleic acid sequence as represented in SEQ ID NO: 5;
iv) a nucleic acid sequence as represented in SEQ ID NO: 7;
v) a nucleic acid sequence as represented in SEQ ID NO:9;
vi) a nucleic acid sequence as represented in SEQ ID NO: 11; or a nucleic acid molecule comprising a nucleic sequence that hybridizes under stringent hybridization conditions to SEQ ID NO: 1, 3, 5, 7, 9 or 11 and which encodes a polypeptide that has insulin-like growth factor modulating activity.

In a preferred embodiment of the invention said nucleic acid molecule encodes a polypeptide that has agonist activity.

In a preferred embodiment of the invention said nucleic acid molecule encodes a polypeptide that has antagonist activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said nucleic acid molecule comprises or consists of a nucleic acid sequence as represented in SEQ ID NO: 1.

In a preferred embodiment of the invention said nucleic acid molecule comprises or consists of a nucleic acid sequence as represented in SEQ ID NO: 3.

In a preferred embodiment of the invention said nucleic acid molecule comprises or consists of a nucleic acid sequence as represented in SEQ ID NO: 5.

In a preferred embodiment of the invention said nucleic acid molecule comprises or consists of a nucleic acid sequence as represented in SEQ ID NO: 7.

In a preferred embodiment of the invention said nucleic acid molecule comprises or consists of a nucleic acid sequence as represented in SEQ ID NO: 9.

In a preferred embodiment of the invention said nucleic acid molecule comprises or consists of a nucleic acid sequence as represented in SEQ ID NO: 11.

According to an aspect of the invention there is provided a polypeptide encoded by the nucleic acid according to the invention.

According to a further aspect of the invention there is provided a polypeptide comprising or consisting of an amino acid sequence selected from the group consisting of: SEQ ID NO: 2, 4, 6, 8, 10, 12, 15, 16, 17, 18, 19 or 20.

According to an aspect of the invention there is provided a homodimer consisting of two polypeptides wherein each of said polypeptides comprises:
i) a first part comprising insulin-like growth factor, or a receptor binding domain thereof, optionally linked by a peptide linking molecule to
ii) a second part comprising at least one insulin-like growth factor binding domain or part thereof, of the insulin-like growth factor receptor.

In a preferred embodiment of the invention said homodimer comprises two polypeptides comprising or consisting of SEQ ID NO: 2, 4, 6, 8, 10, 12, 15, 16, 17, 18, 19 or 20.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a human subject suffering from a disease or condition related to severe primary insulin-like growth factor deficiency comprising administering an effective amount of at least one polypeptide according to the invention.

In a preferred method of the invention said severe primary deficiency is Laron dwarfism.

In a further preferred method of the invention said disease is a disease that does not respond to growth hormone therapy.

In a further preferred method of the invention said disease is type I diabetes.

In an alternative preferred method of the invention said disease is type II diabetes.

In a further preferred method of the invention said disease is amyotrophic lateral sclerosis.

In a further preferred method of the invention said disease is myotonic muscular dystrophy.

In a further preferred method of the invention said condition is severe burn injury.

According to a further aspect of the invention there is provided a method to treat a human subject suffering from cancer comprising administering an effective amount of a polypeptide antagonist according to the invention.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In a preferred method of the invention said polypeptide is administered intravenously.

In an alternative preferred method of the invention said polypeptide is administered subcutaneously.

In a further preferred method of the invention said polypeptide is administered at two day intervals; preferably said polypeptide is administered at weekly, 2 weekly or monthly intervals.

According to a further aspect of the invention there is provided a monoclonal antibody that binds the polypeptide or dimer according to the invention.

Preferably said monoclonal antibody is an antibody that binds the polypeptide or dimer but does not specifically bind insulin-like growth factor or insulin-like growth factor receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention, or fragments thereof;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

According to a further aspect of the invention there is provided a diagnostic test to detect a polypeptide according to the invention in a biological sample comprising:
i) providing an isolated sample to be tested;
ii) contacting said sample with a ligand that binds the polypeptide or dimer according to the invention; and
iii) detecting the binding of said ligand in said sample.

In a preferred embodiment of the invention said ligand is an antibody; preferably a monoclonal antibody.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

Table 1 summary of fusion protein nomenclature;

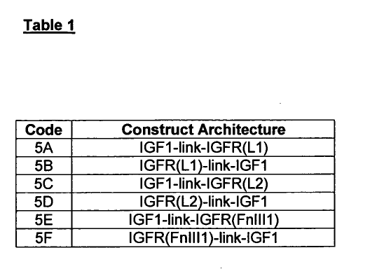

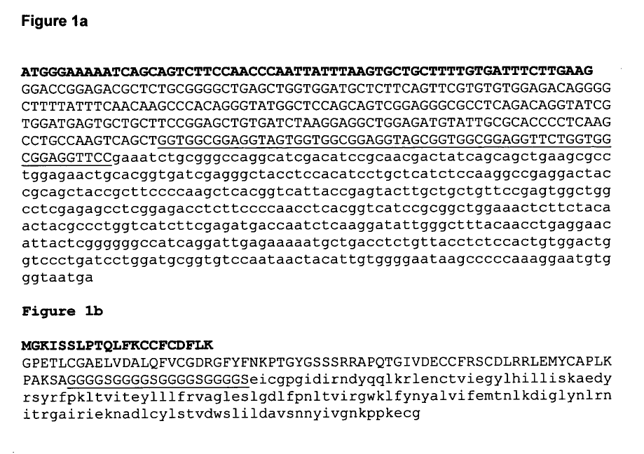

FIG. 1a the nucleic acid sequence of LR 5A1; FIG. 1b is the amino acid sequence of LR5A1;

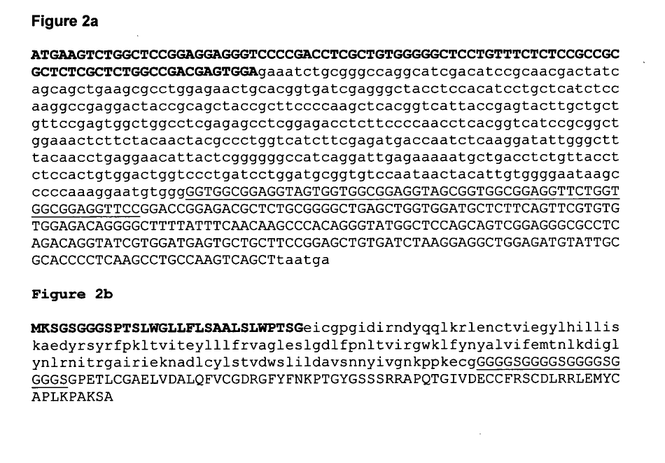

FIG. 2a the nucleic acid sequence of LR 5B1; FIG. 2b is the amino acid sequence of LR5B1;

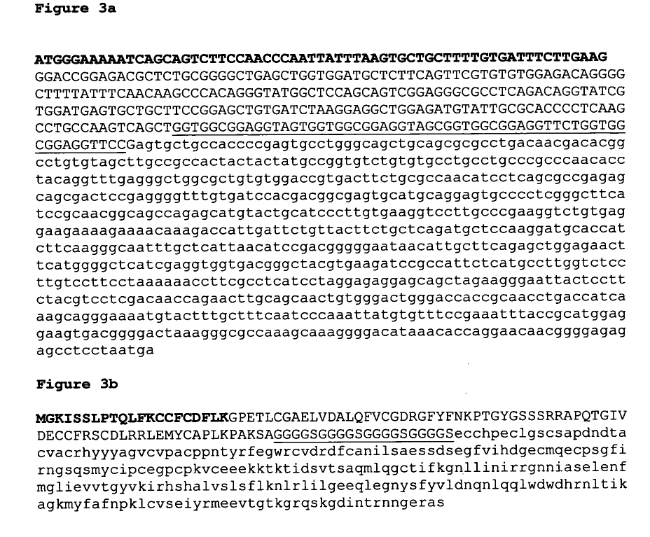

FIG. 3a the nucleic acid sequence of LR 5C1; FIG. 3b is the amino acid sequence of LR5C1;

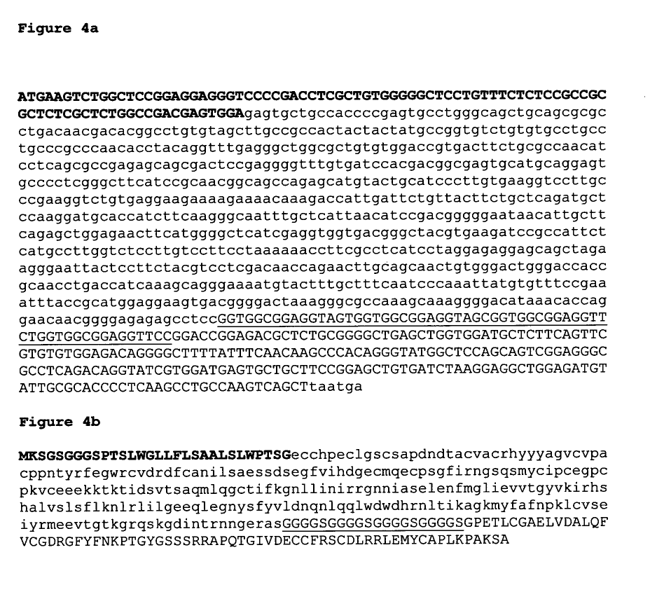

FIG. 4a the nucleic acid sequence of LR 5D1; FIG. 4b is the amino acid sequence of LR5D1;

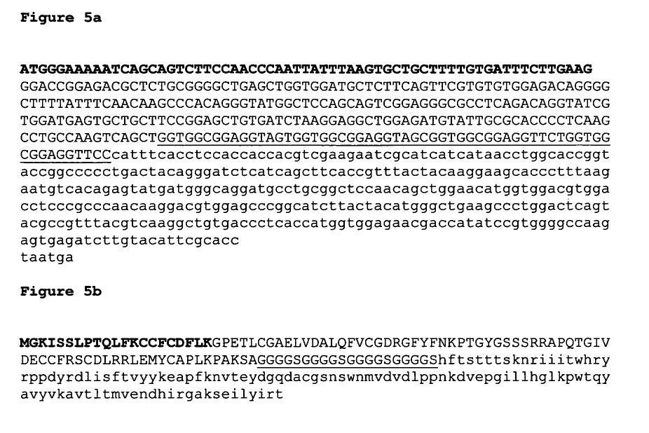

FIG. 5a the nucleic acid sequence of LR 5E1; FIG. 5b is the amino acid sequence of LR5E1;

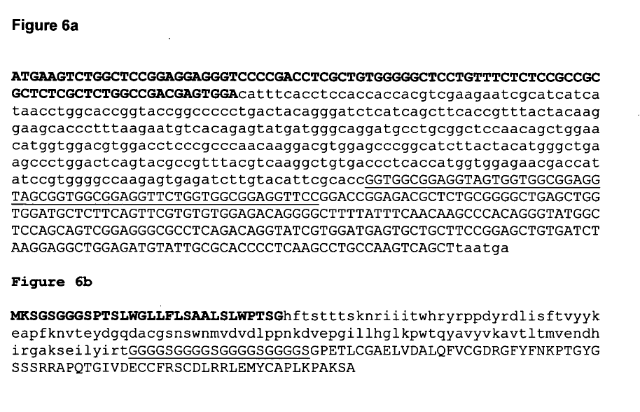

FIG. 6a the nucleic acid sequence of LR 5F1; FIG. 1b is the amino acid sequence of LR5F1;

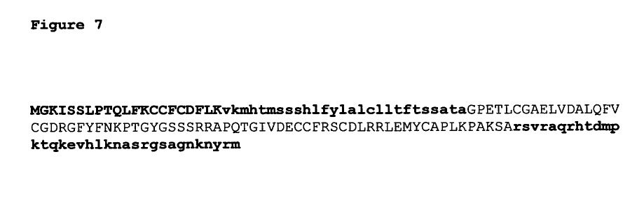

FIG. 7 is the amino acid sequence of human IGF-1 A;

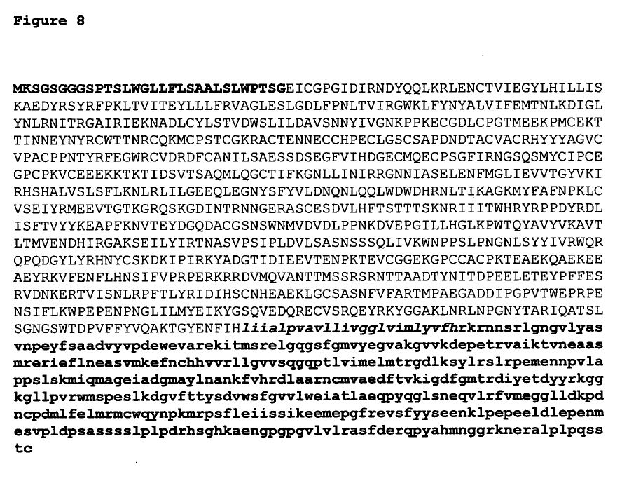

FIG. 8 is the amino acid sequence of human IGF-1 receptor;

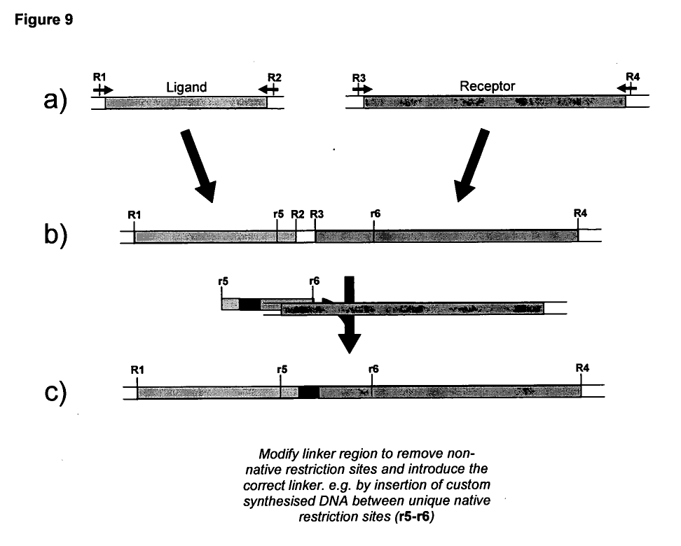

FIG. 9 PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites);

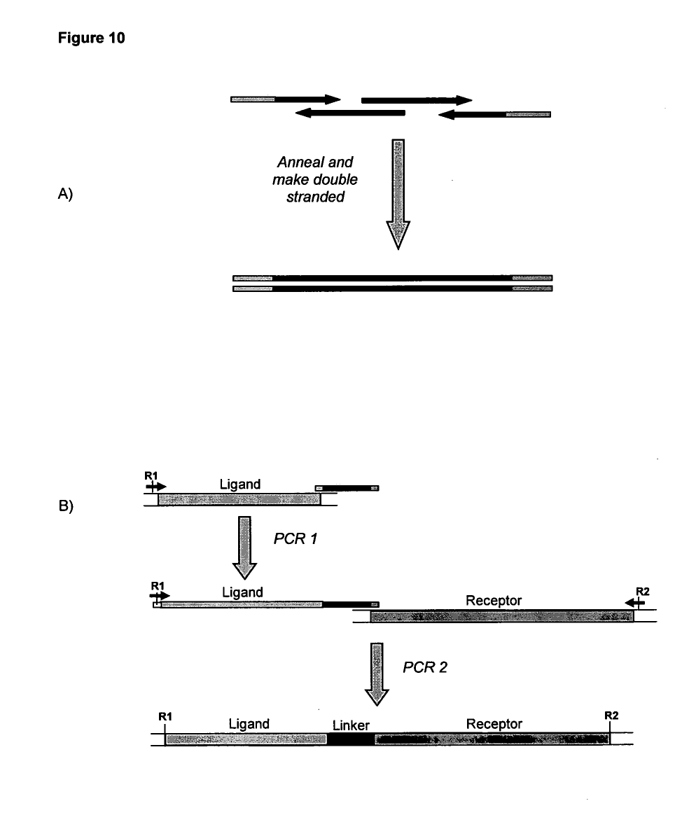

FIG. 10 Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector;

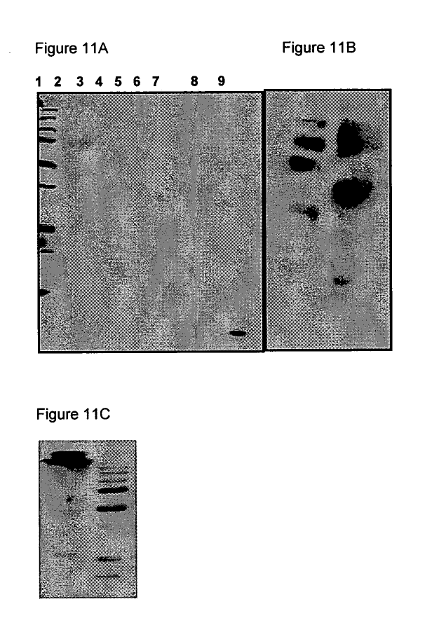

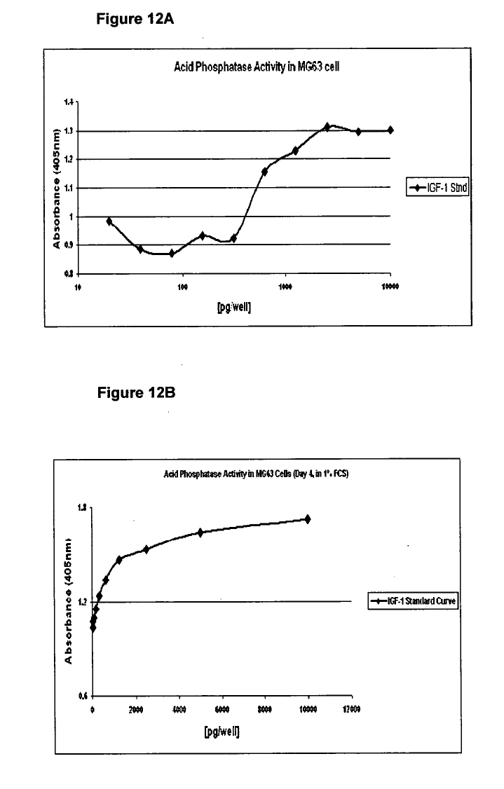

FIG. 11A shows western blot of CHO cell expressed 5A1. Samples were prepared as described in the presence of DTT. Lane 1: Ladder, lane 2: 5A1 (100× concentrated media from stable cell line), Lane 3: 5A1 Transient Transfection (DNA: fugene 6 @ 3:2), Lane 4: 5A1 Transient Transfection (DNA: fugene 6 @ 3:1), Lane 5: 5A1 Transient Transfection (DNA+TransIT), Lane 6: −ve control (100× concentrated media from 1B7stop stable cell line), Lane 7: Positive control, 100 ng rh-IGF-1. FIG. 11B: Shows re-probed western blot of 5A1 at longer exposure times: Lane 1: Standards, lane 2: 5A1 (100× concentrated media from stable cell line) 5A1 separates out as a major doublet band of 75 and 40 kDa. Non glycosylated MW is approximately 28.4 kDa IGF-1 control protein has a MW of 17 kDa. FIG. 11C shows 5A1 media sample run under non reducing conditions. The majority of 5A1 runs above the 250 kDa marker indicating the molecule may be associating via disulphide linkages;

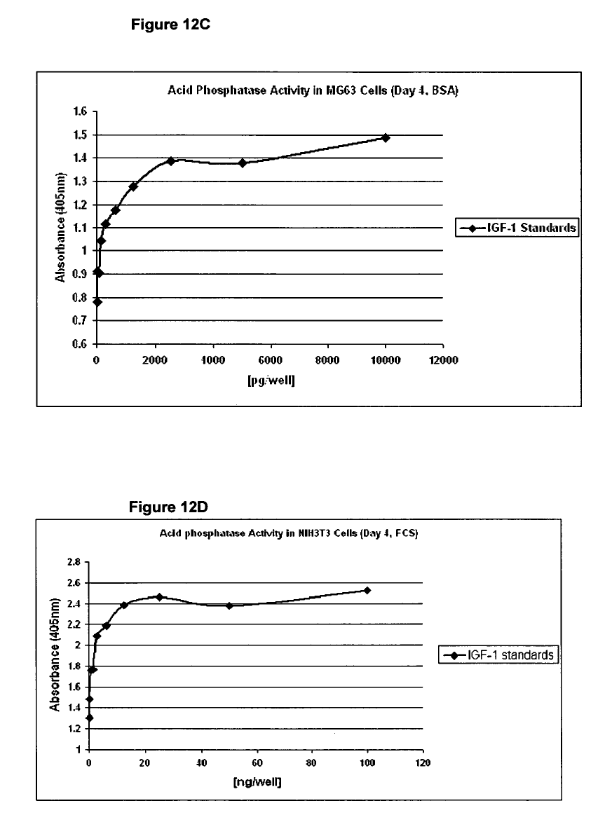

FIG. 12A illustrates the bioactivity of recombinant IGF-1 in stimulating acid phosphatase activity of MG63 cells after 3 days growth in 1% fetal calf serum; FIG. 12B illustrates the bioactivity of recombinant IGF-1 in stimulating acid phosphatase activity of MG63 cells after 4 days growth in 1% fetal calf serum; FIG. 12C illustrates the bioactivity of recombinant IGF-1 in stimulating acid phosphatase activity of MG63 cells after 4 days growth in 2 mg/ml BSA; FIG. 12D illustrates the bioactivity of recombinant IGF-1 in stimulating acid phosphatase activity of NIH3T3 cells after 4 days growth in 1% fetal calf serum; and

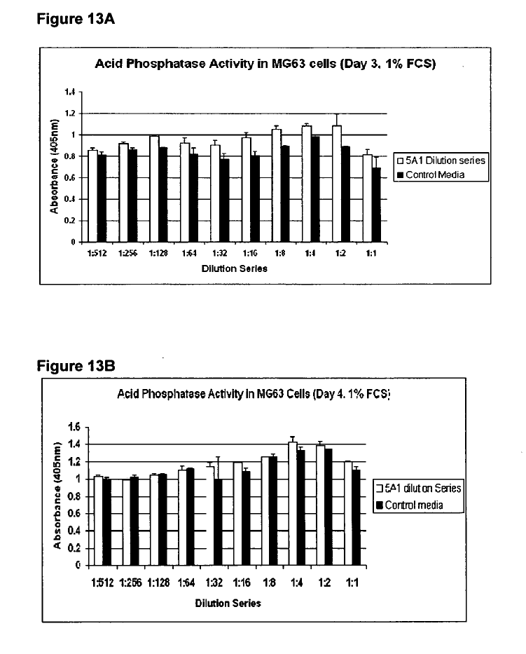

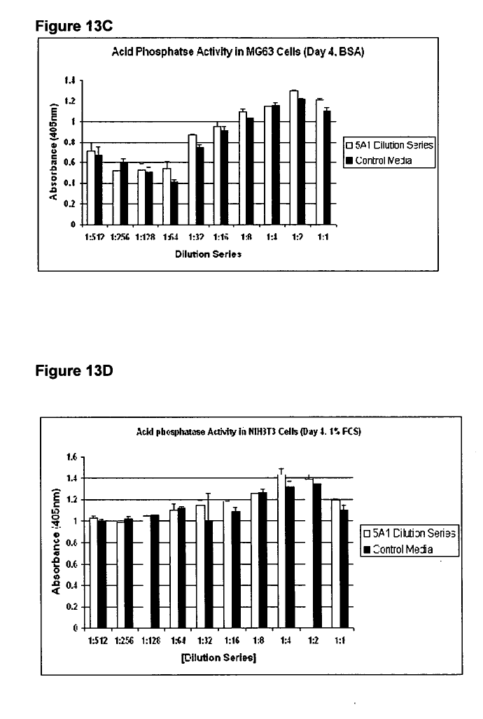

FIG. 13A illustrates a comparison of control medium derived from cells not expressing 5A1 with medium derived from cells expressing 5A1 and serially diluted and their effects on acid phosphatase activity of MG63 cells after 3 days growth in 1% fetal calf serum; FIG. 13B illustrates a comparison of control medium derived from cells not expressing 5A1 with medium derived from cells expressing 5A1 and serially diluted and their effects on acid phosphatase activity of MG63 cells after 4 days growth in 1% fetal calf serum; FIG. 13C illustrates a comparison of control medium derived from cells not expressing 5A1 with medium derived from cells expressing 5A1 and serially diluted and their effects on acid phosphatase activity of MG63 cells after 4 days growth in 2 mg/ml BSA; FIG. 13D illustrates a comparison of control medium derived from cells not expressing 5A1 with medium derived from cells expressing 5A1 and serially diluted and their effects on acid phosphatase activity of NIH3T3 cells after 4 days growth in 1% fetal calf serum.

### MATERIALS AND METHODS

### In Vitro Tests

In vitro tests for detecting and assessing the activity of IGF-1 are known in the art. For example, Pietrzkowski et al (Molecular and Cellular Biology (1992) Vol 12, no 9 p 3883-3889) describes the expression of IGF-1 receptor in BALB 3T3 cells and exposure to IGF-1 results in stimulation cell proliferation and autophosphorylation of IGF-1 receptor. Also see Flier et al (PNAS (1986) 83: 664-668) that describes the blocking of IGF-1 receptor activation by IGF-1 using an antagonistic monoclonal antibody.

### In Vivo Tests

Various animal models of IGF-1 are available to test the activity of IGF-1. For example, Lembo et al (J. Clinical Investigation (1999) 98, 2648-2655) describes a mutant IGF-1 mouse that carries a mutant allele that has a 30% reduction in wild-type IGF-1 levels but were able to survive into adulthood. In Liu et al (Cell (1993) 75: 59-72) and Powell-Braxton et al (Genes and Development (1993) 7: 2609-2617) describe homozygous mice that show severe embryonic and post-natal growth defects.

### Immunological Testing

Immunoassays that measure the binding of IGF-1 to polyclonal and monoclonal antibodies are known in the art. Commercially available IGF-1 antibodies are available to detect IGF-1 in samples and also for use in competitive inhibition studies. For example see http://www.abcam.com/index.html, Abcam PLC.

### Recombinant Production of Fusion Proteins

The components of the fusion proteins were generated by PCR using primers designed to anneal to the ligand or receptor and to introduce suitable restriction sites for cloning into the target vector (FIG. 9a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 9b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 9c).

Alternatively, the linker with flanking sequence, designed to anneal to the ligand or receptor domains of choice, was initially synthesised by creating an oligonucleotide duplex and this processed to generate double-stranded DNA (FIG. 10a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 10b).

### Expression and Purification of Fusion Proteins

Expression was carried out in a suitable system (e.g. mammalian CHO cells, E. coli,) and this was dependant on the vector into which the LR-fusion gene was generated. Expression was then analysed using a variety of methods which could include one or more of SDS-PAGE, Native PAGE, western blotting, ELISA.

Once a suitable level of expression was achieved the LR-fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin). Purified protein was analysed using a variety of methods which could include one or more of Bradford's assay, SDS-PAGE, Native PAGE, western blotting, ELISA.

### Characterisation of LR-Fusions

Denaturing PAGE, native PAGE gels and western blotting were used to analyse the fusion polypeptides and western blotting performed with antibodies non-conformationally sensitive to the LR-fusion. Native solution state molecular weight information can be obtained from techniques such as size exclusion chromoatography using a Superose G200 analytical column and analytical ultracentrifugation.

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

### Construction of Chimeric Clones

All clones were ligated using the restriction enzymes Nhe1/HindIII, into the mammalian expression plasmid pSecTag-link. Clones were attached to the secretion signal for human IGF-1 for efficient secretion into cell media. The whole gene for 5A1 was cloned using gene synthesis and cloned into the mammalian expression vector pSecTag-link to form pIGFsecTag-5A1.

### Mammalian Stable Expression

A mammalian expression system has been established using a modification of the invitrogen vector pSecTag-V5/FRT-Hist

### Invitrogen's Flp-In System

This system allows for the rapid generation of stable clones into specific sites within the host genome for high expression. This can be used with either secreted or cytoplasmic expressed proteins. Flp-In host cell lines (flp-In CHO) have a single Flp recombinase target (FRT) site located at a transcriptionally active genomic locus. Stable cell lines are generated by co-transfection of vector (Containing FRT target site) and pOG44 (a [plasmid that transiently expresses flp recombinase) into Flp-In cell line. Selection is with Hygromycin B. There is no need for clonal selection since integration of DNA is directed. Culturing Flp-In Cell lines: followed manufactures instruction using basic cell culture techniques.

### Stable Transfection of CHO Flp-In Cells Using Fugene-6

The day before transfection CHO Flp-In cells were seeded at 6×10E5 per 100 mm petri dish in a total volume of 10 ml of Hams F12 media containing 10% (v/v) Fetal Calf Serum, 1% Penicillin/streptomycin and 4 mM L-glutamine. The next day added 570 μl of serum free media (containing no antibiotics) to a 1.5 ml polypropylene tube. 30 μl of fugene-6 was then added and mixed by gentle rolling. A separate mix of plasmids was set up for each transfection which combined 2 μg plasmid of interest with 18 μg pOG44 (plasmid contains recombinase enzyme necessary for correct integration of plasmid into host genome). Control plate received no plasmid. This was mixed with fugene-6 by gentle rolling, left @ RmT for 15 minutes, then applied drop-wise to the surface of the each petri dish containing CHO Flp-In cells in F12 media+10% FCS. The plates were gently rolled to ensure good mixing and left for 24 hrs @ 37° C./5% CO2. The next day media was exchanged for selective media containing hygromycin B @ 600 ug/ml. Cells were routinely kept at 60% confluency or less. Cells were left to grow in the presence of 600 ug/ml hygromycin B until control plate cells (non transfected cells) had died (i.e. no hygromycin resistance).

Testing Expression from Stable CHO Cell Lines

Confluent CHO Flp-In cell lines expressing the protein of interest were grown in 75 cm2 flasks for approximately 3-4 days in serum free media, at which point samples were taken and concentrated using acetone precipitation. Samples were mixed with an equal volume of laemmli loading buffer in the presence or absence of 25 mM DTT and boiled for 5 minutes. Samples were analysed by SDS-PAGE and transferred to a PVDF membrane. After blocking in 5% (w/v) Milk protein in PBS-0.05% (v/v) Tween 20, sample detection was carried out using a specific anti-IGF-1 antibody together with a Horse Radish Peroxidase (HRP) conjugated secondary antibody. Visualisation was by chemiluminesence on photographic film using an HRP detection kit.

Testing Expression from Transient Transfections

CHO Flp-In cells were seeded at 0.25×10E6 cells per well of a 6 well plate in a total volume of 2 ml media (DMEM, F12, 10% FCS+P/S+L-glutamine+Zeocin). Cells were left to grow o/n. Cells were then transfected using either TransIT-CHO Reagent (Mirus) or fugene-6 at the specified reagent ratios stated in table 1. Control transfections were set up using 1B7stop (GH containing chimeric molecule). Briefly, if using TransIT reagent, 200 ul of Serum free media (OPTI MEM) was added to a 1.5 ml eppendorff per transfection followed by 2 ug DNA. The tubes were left for 15 minutes at RmT. 1 ul of CHO Mojo Reagent was then added, mixed and left for a further 15 minutes. Media was changed to serum free and the transfection mix pippetted dropwise onto the surface of the appropriate well. Briefly, if using Fugene-6 reagent, 94 ul of Serum free media (OPTI MEM) was added to a 1.5 ml eppendorff per transfection followed by 2 ug DNA. The tubes were left for 15 minutes at RmT. Transfection mix was then pippetted drop wise onto the surface of the appropriate well containing serum free media. All plate were left @ 37 deg C./5% CO2 for 2-3 days.

### IGF-1 Bioactivity Assay

### Assay Medium

MG63 cells: EMEM supplemented with 10% FCS, Pen/Strep, 5 mM L-glutamine, non essential amino acids (NEAA)=complete EMEM NIH 3T3 cells: DMEM+glutamax (4.5 g/L Glucose]+10% FCS, Pen/Strep, 2 mM Sodium pyruvate, 5 mM L-glutamine=complete DMEM.

MG63 cells: EMEM [Gibco] supplemented with 2 mg/ml BSA or 1% FCS, 5 mM L-Glutamine, Pen/Strep, NEAA. NIH 3T3 cells: DMEM+glutamax [Gibco; Cat# 61956, Lot# 357700]+1% FCS (or 2 mg/ml BSA), 5 mM L-Glutamine [Gibco], Pen/Strep [Gibco], 2 mM Sodium pyruvate [Gibco].

### Assay Method

1. For the assay; TE treat cells and count. Adjust density to ˜5×10E3 cells in 50 ul (1×10E5 cells per ml) in DMEM supplemented with 1% FCS (or 2 mg/ml BSA). Plate on a 96 well plate.
2. Prepare a series of IGF-1 dilutions in DMEM (with 1% FCS or 2 mg/ml BSA) and add 50 ul of each dilution (0-100 ng/well) to separate wells containing cells. Do the same for media samples
3. Grow cells in the presence of IGF-1 or Test Samples for 3 and 4 days @ 37C/5% CO2.
4. To Assay: Remove medium from wells and rinse each well once with 200 ul PBS buffer.
5. Assay for alkaline phosphatase using pNPP in Assay buffer: Add 100 ul per well.
6. Incubate @ 37C for 2 hrs.
7. Add 10 ul of 1M NaOH to each well to stop the reaction.
8. Incubate plate at RmT for 5-20 minutes to allow the colour to develop.
9. Record the A405 nm of each well in a microplate reader.

### Controls

1. Assay medium plus substrate (no bioactive factor): determines the amount of non-enzymic hydrolysis of substrate. These values are subtracted from each of the experimental values.
2. Cells only plus substrate (no bioactive factor): this represents how much the cells have grown in the absence of factor.

### Example

The ability of the cell lines MG63 and NIH 3T3 to proliferate in the presence of IGF-1 or chimera (5A1) was tested. The test is based on the assay of endogenous acid phosphatase activity using the substrate p-nitrophenyl phosphate. MG63 cells (human osteosarcoma cell line: Cat# 86051601, lot# 05F008, ECACC) NIH3T3 cells; Mouse fibroblast cell line [Obtained from Simon Smith, ARCBioserv, Sheffield University: Date on vial Jun. 24, 1993]. Both MG63 and NIH 3T3 cells respond well to the presence of recombinant IGF-1 giving a good dose response curve; see FIGS. 12A, 12B, 12C and 12D. These data show that in the presence of 5A1 media the cells proliferate producing a shallow dose response at lower dilutions. 5A1 media sample consistently produces a higher degree of proliferation than that of control medium; see FIG. 13A, 13B, 13C or 13D.

1. (canceled)

2. A fusion polypeptide comprising: the amino acid sequence of insulin-like growth factor polypeptide, or active part thereof linked, directly or indirectly, to at least one insulin-like growth factor polypeptide binding domain of the insulin-like growth factor polypeptide receptor polypeptide.

3. A fusion polypeptide according to claim 2 wherein said polypeptide binding domain comprises a leucine rich amino acid motif.

4-5. (canceled)

6. A fusion polypeptide according to claim 3 wherein said polypeptide comprises at least one fibronectin III binding domain.

7-11. (canceled)

12. A fusion polypeptide according to claim 2 wherein said insulin-like growth factor polypeptide is linked to said binding domain of the insulin-like growth factor receptor polypeptide by a peptide linker.

13-14. (canceled)

15. A fusion polypeptide according to claim 2 wherein said polypeptide does not comprise a peptide linking molecule and is a direct fusion of insulin-like growth factor polypeptide and said binding domain of the insulin-like growth factor receptor polypeptide.

16. An isolated nucleic acid molecule comprising a nucleic acid sequence selected from:
i) a nucleic acid sequence as represented in SEQ ID NO: 1;
ii) a nucleic acid sequence as represented in SEQ ID NO: 3;
iii) a nucleic acid sequence as represented in SEQ ID NO: 5;
iv) a nucleic acid sequence as represented in SEQ ID NO: 7;
v) a nucleic acid sequence as represented in SEQ ID NO: 9;
vi) a nucleic acid sequence as represented in SEQ ID NO: 11 and
vii) a nucleic acid sequence that hybridizes under stringent hybridization conditions to SEQ ID NO: 1, 3, 5, 7, 9 or 11 and which encodes a polypeptide that has insulin-like growth factor modulating activity.

17-24. (canceled)

25. An isolated polypeptide encoded by the nucleic acid molecule according to claim 16.

26. An isolated polypeptide comprising an amino acid sequence selected from the group consisting of: SEQ ID NO: 2, 4, 6, 8, 10, 12, 15, 16, 17, 18, 19 and 20.

27. A homodimer consisting of two polypeptides wherein each of said polypeptides comprises:
i) a first part comprising insulin-like growth factor, or a receptor binding domain thereof, and
ii) a second part comprising at least one insulin-like growth factor binding domain or part thereof, of the insulin-like growth factor receptor.

28. A homodimer according to claim 27 wherein said homodimer comprises two polypeptides comprising SEQ ID NO: 2, 4, 6, 8, 10, 12, 15, 16, 17, 18, 19 or 20.

29. A vector comprising a nucleic acid molecule according to claim 16.

30. A cell transfected or transformed with a nucleic acid molecule according to claim 16.

31-32. (canceled)

33. A pharmaceutical composition comprising a polypeptide according to claim 2 and an excipient or carrier.

34. (canceled)

35. A method to treat a human subject suffering from a disease or condition related to severe primary insulin-like growth factor deficiency comprising administering an effective amount of at least one polypeptide according to claim 2.

36. A method according to claim 35 wherein said severe primary deficiency is Laron dwarfism.

37. A method according to claim 35 wherein said disease is a disease that does not respond to growth hormone therapy.

38. A method according to claim 35 wherein said disease or condition is selected from the group consisting of: type I diabetes; type II diabetes; amyotrophic lateral sclerosis; myotonic muscular dystrophy; and severe burn injury.

39-57. (canceled)


SEQUENCE LISTING (21 sequences)

SEQ ID NO: 1 - DNA, 786 bp, Artificial
  IGF-1/IGF receptor fusion protein
atgggaaaaatcagcagtcttccaacccaattatttaagtgctgcttttgtgatttcttgaagggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagctggtggcggaggtagtggtggcggaggtagcggtggcggaggttctggtggcggaggttccgaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtgggtaatga

SEQ ID NO: 2 - protein, 260 aa, Artificial
  IGF-1/IGF-1 receptor fusion protein
MGKISSLPTQLFKCCFCDFLKGPETLCGAELVDALQFVCGDRGFYFNKPTGYGSSSRRAPQTGIVDECCFRSCDLRRLEMYCAPLKPAKSAGGGGSGGGGSGGGGSGGGGSEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECG

SEQ ID NO: 3 - DNA, 813 bp, Artificial
  IGF-1/IGF receptor fusion protein
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtgggggtggcggaggtagtggtggcggaggtagcggtggcggaggttctggtggcggaggttccggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagcttaatga

SEQ ID NO: 4 - protein, 269 aa, Artificial
  IGF-1/IGF-1 receptor fusion protein
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGGGGGSGGGGSGGGGSGGGGSGPETLCGAELVDALQFVCGDRGFYFNKPTGYGSSSRRAPQTGIVDECCFRSCDLRRLEMYCAPLKPAKSA

SEQ ID NO: 5 - DNA, 1116 bp, Artificial
  IGF-1/IGF-1 receptor fusion protein
atgggaaaaatcagcagtcttccaacccaattatttaagtgctgcttttgtgatttcttgaagggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagctggtggcggaggtagtggtggcggaggtagcggtggcggaggttctggtggcggaggttccgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctaatga

SEQ ID NO: 6 - protein, 370 aa, Artificial
  IGF-1/IGF receptor fusion protein
MGKISSLPTQLFKCCFCDFLKGPETLCGAELVDALQFVCGDRGFYFNKPTGYGSSSRRAPQTGIVDECCFRSCDLRRLEMYCAPLKPAKSAGGGGSGGGGSGGGGSGGGGSECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERAS

SEQ ID NO: 7 - DNA, 1143 bp, Artificial
  IGF-1/IGF-1 receptor fusion protein
atgaagtctggctccggaggagggtccccgacctcgctgtgggggctcctgtttctctccgccgcgctctcgctctggccgacgagtggagagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctccggtggcggaggtagtggtggcggaggtagcggtggcggaggttctggtggcggaggttccggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagcttaatga

SEQ ID NO: 8 - protein, 379 aa, Artificial
  IGF-1/IGF-1 receptor fusion protein
MKSGSGGGSPTSLWGLLFLSAALSLWPTSGECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASGGGGSGGGGSGGGGSGGGGSGPETLCGAELVDALQFVCGDRGFYFNKPTGYGSSSRRAPQTGIVDECCFRSCDLRRLEMYCAPLKPAKSA

SEQ ID NO: 9 - DNA, 678 bp, Artificial
  IGF-1/IGF-1 receptor fusion protein
atgggaaaaatcagcagtcttccaacccaattatttaagtgctgcttttgtgatttcttgaagggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagctggtggcggaggtagtggtggcggaggtagcggtggcggaggttctggtggcggaggttcccatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcacctaatga

SEQ ID NO: 10 - protein, 224 aa, Artificial
  IGF-1/IGF-1 receptor fusion protein
MGKISSLPTQLFKCCFCDFLKGPETLCGAELVDALQFVCGDRGFYFNKPTGYGSSSRRAPQTGIVDECCFRSCDLRRLEMYCAPLKPAKSAGGGGSGGGGSGGGGSGGGGSHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRT

SEQ ID NO: 11 - DNA, 705 bp, Artificial
  IGF-1/IGF-1 receptor fusion protein
atgaagtctggctccggaggagggtccccgacctcgctgtgggggctcctgtttctctccgccgcgctctcgctctggccgacgagtggacatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccggtggcggaggtagtggtggcggaggtagcggtggcggaggttctggtggcggaggttccggaccggagacgctctgcggggctgagctggtggatgctcttcagttcgtgtgtggagacaggggcttttatttcaacaagcccacagggtatggctccagcagtcggagggcgcctcagacaggtatcgtggatgagtgctgcttccggagctgtgatctaaggaggctggagatgtattgcgcacccctcaagcctgccaagtcagcttaatga

SEQ ID NO: 12 - protein, 233 aa, Artificial
  IGF-1/IGF-1 receptor fusion protein
MKSGSGGGSPTSLWGLLFLSAALSLWPTSGHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTGGGGSGGGGSGGGGSGGGGSGPETLCGAELVDALQFVCGDRGFYFNKPTGYGSSSRRAPQTGIVDECCFRSCDLRRLEMYCAPLKPAKSA

SEQ ID NO: 13 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 14 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 15 - protein, 239 aa, Artificial
  IGF-1/IGF-1 receptor fusion protein
GPETLCGAELVDALQFVCGDRGFYFNKPTGYGSSSRRAPQTGIVDECCFRSCDLRRLEMYCAPLKPAKSAGGGGSGGGGSGGGGSGGGGSEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECG

SEQ ID NO: 16 - protein, 239 aa, Artificial
  IGF-1/IGF-1 receptor fusion protein
EICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGGGGGSGGGGSGGGGSGGGGSGPETLCGAELVDALQFVCGDRGFYFNKPTGYGSSSRRAPQTGIVDECCFRSCDLRRLEMYCAPLKPAKSA

SEQ ID NO: 17 - protein, 349 aa, Artificial
  IGF-1/IGF-1 receptor fusion protein
GPETLCGAELVDALQFVCGDRGFYFNKPTGYGSSSRRAPQTGIVDECCFRSCDLRRLEMYCAPLKPAKSAGGGGSGGGGSGGGGSGGGGSECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERAS

SEQ ID NO: 18 - protein, 349 aa, Artificial
  IGF-1/IGF-1 receptor fusion protein
ECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASGGGGSGGGGSGGGGSGGGGSGPETLCGAELVDALQFVCGDRGFYFNKPTGYGSSSRRAPQTGIVDECCFRSCDLRRLEMYCAPLKPAKSA

SEQ ID NO: 19 - protein, 203 aa, Artificial
  IGF-1/IGF-1 receptor fusion protein
GPETLCGAELVDALQFVCGDRGFYFNKPTGYGSSSRRAPQTGIVDECCFRSCDLRRLEMYCAPLKPAKSAGGGGSGGGGSGGGGSGGGGSHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRT

SEQ ID NO: 20 - protein, 203 aa, Artificial
  IGF-1/IGF-1 receptor fusion
HFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTGGGGSGGGGSGGGGSGGGGSGPETLCGAELVDALQFVCGDRGFYFNKPTGYGSSSRRAPQTGIVDECCFRSCDLRRLEMYCAPLKPAKSA

SEQ ID NO: 21 - protein, 5 aa, Artificial Sequence
  Made in a lab
GGGGS